(19) United States
(12) Patent Application Publication
(10) Pub. No.: US 2006/0009673 A1
(43) Pub. Date: Jan. 12, 2006

(54) Ligature weaved mesh tape

(76) Inventor: Wen-Liang Chan, Taipei City (TW)

Correspondence Address: VOLPE AND KOENIG, P.C., UNITED PLAZA, SUITE 1600, 30 SOUTH 17TH STREET, PHILADELPHIA, PA, 19103, US

(21) Appl. No.: 10/885,341

(22) Filed: Jul. 6, 2004

(51) Int. Cl. A61F 2/00

(52) U.S. Cl. 600/29

(57) ABSTRACT

A surgical mesh tape sling is provided in the present invention. The surgical mesh tape sling includes a mesh and a holding ligature freely passing through the mesh. The holding ligature is weaved into the mesh without any affixed knot on the mesh and is capable of moving in body's tissue. In addition, when the present mesh tape sling is used in anti-SUI surgery, the both mechanisms of dynamic kinking effect and automatic compression onto the urethra in one sling operation would be provided thereby. Therefore, the present mesh tape sling is able to treat both categories of SUI, “SUI without intrinsic sphincter deficiency” and “SUI with intrinsic sphincter deficiency”.

### FIELD OF THE INVENTION

The present invention relates to a mesh tape, and more particular to a mesh tape sling including a mesh tape and at least one moveable ligature.

### BACKGROUND OF THE INVENTION

All over the world, the suburethral sling operations including the conventional suburethral sling operation and the new technique of tension-free vaginal tape (TVT) operation are common primary surgeries for the patients with stress urinary incontinence (SUI) at present. In the TVT technique, a mesh tape is applied to reinforce the pubourethral ligament and improve the hammock-like effect on the midurethra. It is mainly suitable for the patients with urethral hypermobility and mild degrees of intrinsic sphincter deficiency (ISD). However, the TVT technique does not satisfy patients with poor degrees of ISD.

Conventionally, the ligature used in the suburethral sling anti-SUI procedure is placed from the urethra to the abdominal wall and formed a complete loop to hold both the urethra and the abdominal wall. The force of the abdominal wall movement during coughing or straining will tense the loop and cause the urethral compression which prevents urine leakage from the bladder. The concept of urethral compression had been used in the cure for the SUI patients with ISD. The conventional ligature sling is suitable to treat most patients having stress urinary incontinence with or without ISD even suffered from severe ISD but is not so good for the patients who has the abdominal straining during voiding.

Please refer to FIG. 1, which shows the schematic diagrams of the suburethral mesh slings according to the prior art. As shown in FIG. 1, the suburethral mesh sling S₁ₐ includes just a biocompatible mesh, and the suburethral mesh sling S_{1b} is composed of the mesh tape 1 and the ligature 2. Wherein the ligature 2 is affixed onto the mesh tape 1 via the knot 21 and used to keep the mesh tape 1 from stretching deformity during the surgical procedure.

Please refer to FIG. 2, which is a schematic diagram of a suburethral ligature sling according to the prior art. As shown in FIG. 2, the suburethral ligature sling S₂ is composed of a piece of rectangular mesh tape 1 and two ligatures 2. The ligatures 2 are weaved into the ends of the rectangular mesh via helical bites 22.

Please refer to FIGS. 3-7, which are the schematic diagrams showing the steps of the suburethral mesh sling anti-SUI procedure according to the prior art.

Please refer to FIG. 3, wherein the pubic region 3, the finger of the surgeon 4, the clitoris 5, the urethral meatus 6, the vaginal incision 7, the labia minora 8, the suprapubic region 9, the anus 10, the Foley catheter 11 and the anterior vaginal wall 12 are shown. In addition, please refer to FIG. 4 showing the pubic region 3, the finger of a surgeon 4, the clitoris 5, the urethral meatus 6, the vaginal incision 7, the labia minora 8, the suprapubic region 9, the anus 10, the Foley catheter 11, the anterior vaginal wall 12, the suburethral mesh sling Sla, two taping wounds 91 and 92 made over the suprapubic region 9 and at about 1-2 cm above the upper margins of the pubic symphysis (not shown), the carrier needle 14 and the needle eye 15 are also shown. In general, a 2.5 cm longitudinal vaginal incision 7 is made over the middle line of the anterior vaginal wall 12 and 1 cm away from the urethral meatus 6. The labia minora 8 is stitched laterally. The anterior vaginal wall 12 is dissected bilaterally and the finger of the surgeon 4 is able to insert into the paraurethral spaces through the vaginal incision 7 to guide the direction of the carrier needle 14. Since the method of how the suburethral mesh sling S₁ₐ is placed into the retropubic space is the same as that of the prior art, the relevant details are omitted for simplification. The carrier needle 14 is placed into the retropubic space from the suprapubic skin taping wound 91 or 92 to the vaginal incision 7, and the suburethral mesh sling S₁ₐ is brought out of the suprapubic skin taping wound 91 and 92 by the carrier needle 14 respectively. In addition, it should be noted that it is possible to replace the suburethral mesh sling S₁ₐ by the suburethral mesh sling S_{1b}, if necessary.

Please refer to FIG. 5, which is the schematic diagram showing the placement of the suburethral ligature sling S₂ in the surgery. The transverse suprapubic skin incision 13 about 3-4 cm in length is made over the suprapubic region 9 and is about 2 cm above the upper margins of the pubic symphysis (not shown). The carrier needle 14 is placed into the retropubic space from the suprapubic skin incision 13 to the vaginal incision 7, and the ligatures 2 are brought out of the suprapubic skin incision 13 by the carrier needle 14. Furthermore, the ligatures 2 of the suburethral sling S₂ are tied together above the rectus fascia via the skin incision 13.

Please refer to FIG. 6, wherein the suburethral mesh sling S₁ₐ has been brought out of the suprapubic skin taping wounds 91 and 92. The tension of the suburethral mesh sling S₁ₐ is adjusted so as that it is allowed to easily place a scissor tip 16 between the suburethral mesh sling S₁ₐ and the urethra (not shown). The distance between the suburethral mesh sling S₁ₐ and the urethra is about 1-2 mm. Then the redundant suburethral mesh sling S₁ₐ is trimmed off at the level of abdominal skin (not shown). Conventionally, the zigzag margins of the tape of the suburethral mesh sling S₁ₐ can be spontaneously hold and secured by the surrounding tissue without sutures or bites. In addition, it should be noted that it is possible to replace the suburethral mesh sling S₁ₐ by the suburethral mesh sling S_{1b}, if necessary.

Please refer to FIG. 7, wherein a piece of rectangular mesh 17 is weaved by the ligature 2 of the mesh sling S₂ and then placed into the suprapubic skin incision 13 above the rectus fascia (not shown). The ligatures 2 of the mesh sling S₂ are tied together after adjusting the tension of mesh sling S₂ either through a coughing test when the procedure was performed under local anesthesia and patient awake or based on surgeon experiences when the operation was performed under general anesthesia. The knots 18 can be placed at the middle or the lateral site of the rectangular mesh 17, wherein the rectangular mesh 17 is placed above the rectus fascia.

Please refer to FIGS. 8 and 9, which are the schematic diagrams showing the steps of the transobturator suburethral mesh sling anti-SUI procedure according to the prior art. In FIG. 8, the pubic region 3, the finger of the surgeon 4, the clitoris 5, the urethral meatus 6, the labia minora 8, the anus 110, the Foley catheter 11, the anterior vaginal wall 12, the skin incision over the genito-femoral fold 20, the curved carrier needle 21. The size of the skin incision over the genito-femoral fold 20 is about 1 cm and the skin incision over the genito-femoral fold 20 is about located at the outer margins of the labia majora (not shown). The curved carrier needle 21 is inserted into the skin incision over the genito-femoral fold 20 and perforated through the obturator membrane (not shown) and muscles (not shown) along the internal edge of the puboischeal ramus (not shown). Then it is facilitated for the curved carrier needle 21 to move forward along the posterior surface of the puboischeal ramus to the vagina. In addition, the finger of the surgeon 4 is inserted into the paraurethral space from the vaginal incision 8 to guide the curved carrier needle 21.

Please refer to FIG. 9, wherein the suburethral mesh sling S₁ₐ is brought out of the skin incision over the genito-femoral fold 20 by the curved carrier needle (not shown) to construct a transobturator suburethral mesh sling. Then both ends of the redundant suburethral mesh sling S₁ₐ are trimmed off at the skin level (not shown). The zigzag margins of the tape of the suburethral mesh sling S₁ₐ can be spontaneously hold and secured by the surrounding tissue and further sutures or bites for fixation are not necessary. In addition, it should be noted that it is possible to replace the suburethral mesh sling S₁ₐ by the suburethral mesh sling S_{1b}, if necessary.

Please refer to FIG. 10, which is the schematic diagram showing the mechanism of the suburethral ligature sling S₂ in the anti-SUI procedure according to the prior art. As shown in FIG. 10, it should be easily understood that the non-adherent monofilament ligature 2 in the suburethral ligature sling S₂ will be pulled upward automatically along with the abdominal wall elevation and the urethral compression is caused during coughing or abdominal straining. In FIG. 10, the reference numeral 24 indicates the position of the ligature 2 of the suburethral ligature sling S₂ at resting, and reference numeral 25 indicates the position of the ligature 2 during coughing or abdominal straining. In addition, the urethral meatus 6, the anterior vaginal wall 12, the urethra 31, the bladder 32, the pubic symphysis 35, and the rectus fascia 36 are also illustrated in FIG. 10.

Please refer to FIG. 11, which is the schematic diagram showing the operation mechanism of the subyrethral mesh sling in the anti-SUI procedure according to the prior art. As shown in FIG. 11, the suburethral mesh sling S₁ₐ or the suburethral mesh sling S_{1b} in FIG. 1 would be adhered and integrated with the surrounding tissue and formed a hammock-like structure to support the urethra 31. And, the dynamic kinking effect will be created at the midurethra during coughing or abdominal straining. In FIG. 11, the proximal portion of the urethra 31 and the bladder 32 would be pushed downward by the forces of the intra-abdominal pressure 37 and become to a temporary kinking deformed urethra 311.

Although the suburethral mesh slings S₁ₐ and S_{1b} (FIG. 1) and the suburethral ligature sling S₂ (FIG. 2) have been widely used in the relevant operations, the clinical effects are still not so good due to the structural drawbacks. The suburethral mesh slings S₁ₐ and S_{1b} are mainly suitable for the patients with urethral hypermobility and mild degree of intrinsic sphincter deficiency (ISD), but do not satisfy patients with poor degree of ISD. The suburethral ligature sling S₂ is suitable to treat the most patients having stress urinary incontinence with or without ISD even suffered from severe ISD but is not so good for the patient who has the abdominal straining during voiding.

The suburethral mesh slings S₁ₐ and S_{1b} would be adhered and integrated into the surrounding tissues while time passes and form a hammock-like structure providing the dynamic kinking effect on the midurethra during coughing or abdominal straining (refer to FIG. 11). On the other hand, since the ligatures 2 of the suburethral ligature sling S₂ are moveable as they are made from the non-absorbable monofilament materials and not adhered with the surrounding tissues, the ligatures 2 of the suburethral ligature sling 2 will be pulled upward during abdominal straining and resulted in an automatic compression effect onto the urethra. (refer to FIG. 10).

In view of the aforesaid discussions, a new ligature weaved mesh tape with longer lifespan and suitable for the cure for the SUI patients even with severe ISD is indeed expected at present.

### SUMMARY OF THE INVENTION

In accordance with one aspect of the present invention, a surgical mesh tape is provided. The surgical mesh tape includes a mesh and a holding ligature weaved into the mesh and capable of freely moving through said mesh.

Preferably, the mesh is made of a biocompatible material.

Preferably, the holding ligature is made of a monofilament of non-absorbable and non-adherent material.

Preferably, the holding ligature is weaved into the mesh and without an affixed knot on the mesh.

Preferably, the holding ligature is weaved into the mesh from one end of the mesh to the other opposite end of the mesh.

Preferably, the holding ligature is weaved into the mesh from a end of the mesh to a middle portion of the mesh and returned back to the end of the mesh.

Preferably, a central portion of the mesh is marked by a color or a special structure.

Preferably, the surgical tape is able of being delivered into a human body for a treatment purpose through different methods.

Preferably, the surgical mesh tape is capable of being used for a patient having a stress urinary incontinence (SUI) and combined with an intrinsic sphincter deficiency (ISD).

Preferably, the surgical mesh tape is capable of being used for a patient having stress urinary incontinence (SUI) but no serve intrinsic sphincter deficiency (ISD).

Preferably, the surgical mesh tape further includes a cover covering the mesh.

Preferably, the cover is a removable cover.

Preferably, the cover is used for positioning the mesh sling.

In accordance with another aspect of the present invention, another surgical mesh tape is provided. The surgical mesh tape includes a mesh sling, at least a ligature weaved into the mesh sling and capable of freely moving through the mesh sling, and an embedded ligature weaved into the mesh sling with affixed knots on the mesh.

Preferably, the embedded ligature is used for keeping the mesh sling from stretching deformity during a surgical procedure.

Preferably, the surgical mesh tape further includes a cover covering the mesh sling.

In accordance with another respect of the present invention, another surgical mesh tape is also provided. The surgical mesh tape includes a mesh sling, a cover covering the mesh sling; and a ligature weaved into the mesh sling with an affixed knot on a central portion of the mesh sling.

Preferably, the central portion has a width ranged from 1 mm to 50 mm.

The above contents and the advantages of the present invention will become more readily apparent to those ordinarily skilled in the art after reviewing the following detailed descriptions and accompanying drawings, in which:

### BRIEF DESCRIPTION OF THE DRAWINGS

FIG. 1 is a conventional suburethral mesh sling according to the prior art;

FIG. 2 is a conventional suburethral ligature sling according to the prior art;

FIGS. 3-7 are the schematic diagrams showing the steps of the suburethral mesh sling anti-SUI procedures according to the prior art;

FIGS. 8-9 are the schematic diagrams showing the steps of the transobturator suburethral mesh sling anti-SUI procedures according to the prior art;

FIG. 10 is the schematic diagram showing the mechanism of the suburethral ligature sling in the anti-SUI procedure according to the prior art;

FIG. 11 is the schematic diagram showing the mechanism of the suburethral mesh sling in the anti-SUI procedure according to the prior art;

FIGS. 12 (a) and (b) are schematic diagrams of the top view and the lateral view of the surgical mesh tape sling S₃ according to the first preferred embodiment of the present invention;

FIGS. 13 (a) and (b) are schematic diagrams of the top view and the lateral view of the surgical mesh tape sling S₄ according to the second preferred embodiment of the present invention;

FIGS. 14 (a) and (b) are schematic diagrams of the top view and the lateral view of the surgical mesh tape sling S₅ according to the third preferred embodiment of the present invention;

FIGS. 15 (a) and (b) are schematic diagrams of the top view and the lateral view of the surgical mesh tape sling S₆ according to the fourth preferred embodiment of the present invention;

FIGS. 16 and 17 are the schematic diagrams showing the application of the surgical mesh tape sling S₃ in the conventional suburethral sling anti-SUI procedure according to a preferred embodiment of the present invention;

FIGS. 18 and 19 are the schematic diagrams showing the application of the surgical mesh tape sling S₅ in the transoburator suburethral sling anti-SUI procedure according to a preferred embodiment of the present invention.

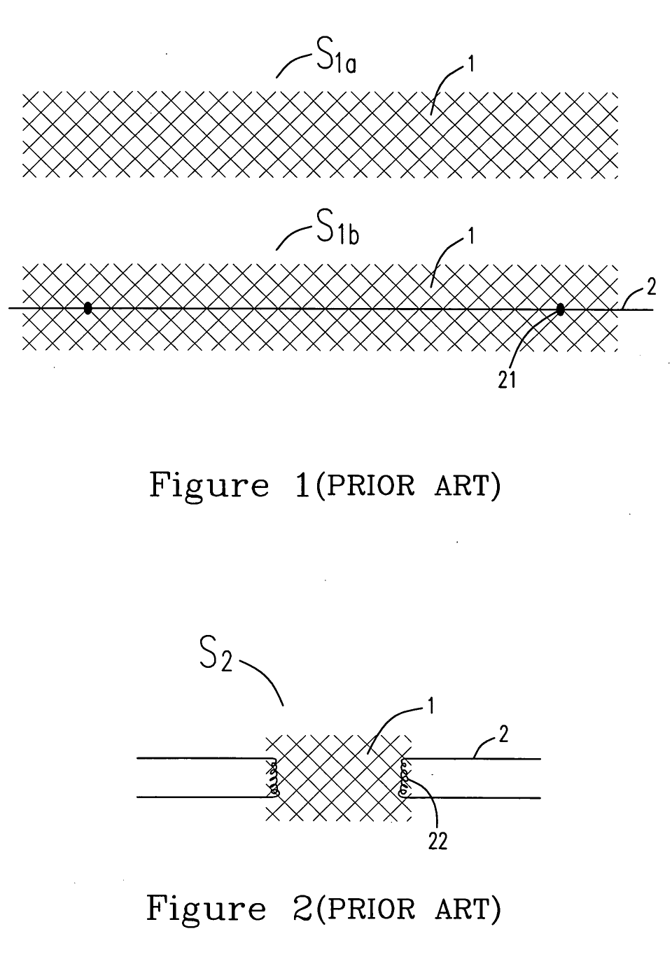

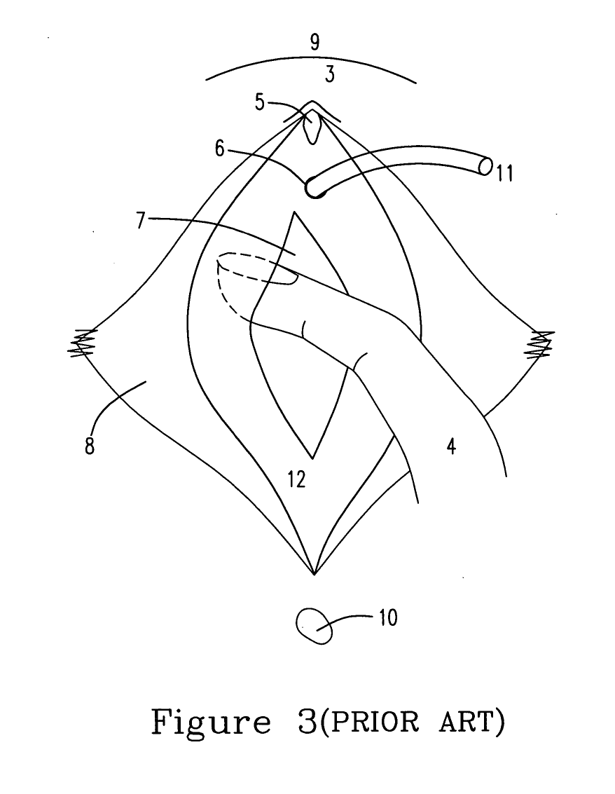

### DETAILED DESCRIPTION OF THE PREFERRED EMBODIMENT

The present invention will now be described more specifically with reference to the following embodiments. It is to be noted that the following descriptions of preferred embodiments of this invention are presented herein for purpose of illustration and description only; it is not intended to be exhaustive or to be limited to the precise form disclosed.

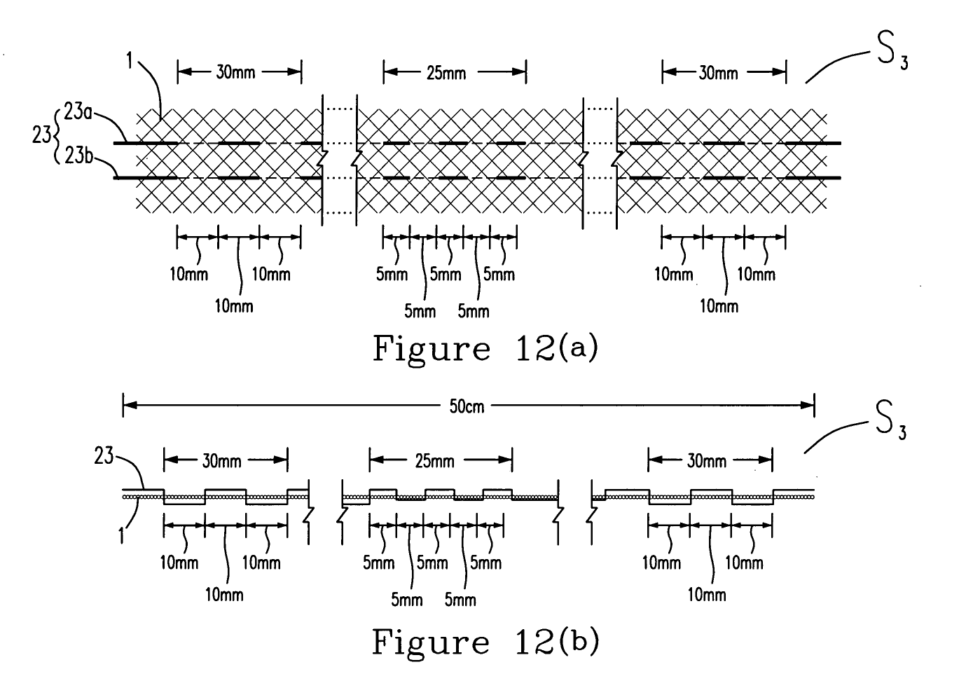

Please refer to FIGS. 12 (a) and (b), which are the schematic diagrams of the top view and the lateral view of the surgical mesh tape sling S₃ according to the first preferred embodiment of the present invention. As shown in FIGS. 12 (a) and (b), the surgical mesh tape sling S₃ according to the first embodiment of the present invention are composed of the mesh tape 1 and the ligature 23 (including the ligatures 23a and 23b). The mesh tape 1 is made of a biocompatible material. The ligatures 23a and 23b are made of non-absorbable and non-adherent monofilament polypropylene, weaved into several sites of the mesh tape 1 with synchronous paths and without any affixed knots, and parallel to the long axis of the mesh tape 1. The size of the mesh tape 1 is 10 mm in width and about 50 cm in length. In the middle portion of the mesh tape 1, the ligatures 23a and 23b are weaved in and out, wherein each interval distance of the ligature weaves is about 5 mm. In the end portions of the mesh tape 1, each interval distance of the ligature weaves is about 1 cm. Since there are no knots between the mesh tape 1 and the ligatures 23a and 23b, and the ligatures 23a and 23b are weaved into the mesh tape 1, the ligatures 23a and 23b are capable of freely moving along with the elevation of the abdominal wall in the body when coughing or abdominal straining. Accordingly, since there is no knot to affix the ligatures 23a and 23b onto the mesh tape 1 in the preferred embodiment of the present invention, the structural drawbacks of the prior mesh sling are overcome. In addition, in order to position the mesh tape sling S₃ more easily, the central portion (such as the portion with the width of 25 mm shown in FIGS. 12 (a) and (b)) of the mesh tape sling S₃ might be colored or constructed with specific structures and dimensions. Further more, it also should be noted that it is also possible that the ligatures 23a and 23b might be affixed onto the central portion of the mesh tape 1 for handling easier, if necessary.

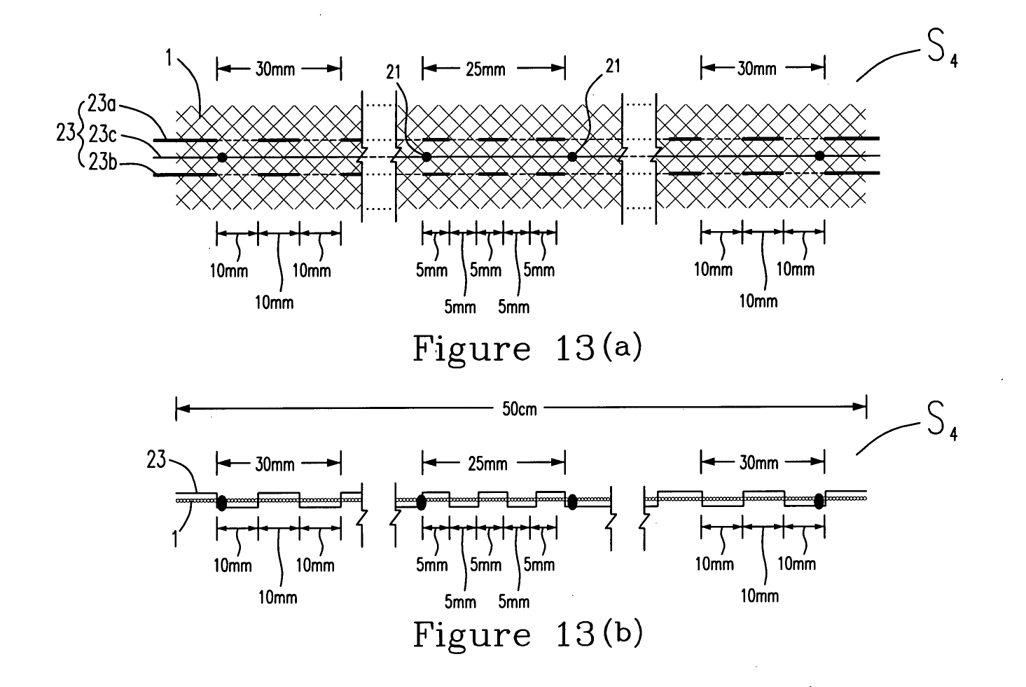

Please refer to FIGS. 13 (a) and (b), which are the schematic diagrams of the top view and the lateral view of the surgical mesh tape sling S₄ according to the second preferred embodiment of the present invention. As shown in FIGS. 13 (a) and (b), the surgical mesh tape sling S₄ according to the second embodiment of the present invention includes the mesh tape 1 and the ligature 23 (including the ligatures 23a, 23b and 23c). The mesh tape 1 is made of a biocompatible material. The ligatures 23a and 23b are made of non-absorbable and non-adherent monofilament polypropylene, weaved into several sites of the mesh tape 1 with synchronous paths and without any affixed knots, and parallel to the mesh tape 1. The ligature 23c is embedded into the mesh tape 1 and with affixed knots 231 on the mesh tape 1 for keeping the mesh tape 1 from stretching deformity during a surgical procedure. In addition, in order to position the mesh tape sling S₄ more easily, the central portion (such as the portion with the width of 25 mm shown in FIGS. 13 (a) and (b)) of the mesh tape sling S₄ might be colored or constructed with specific structures and dimensions. Further more, it also should be noted that it is also possible that the ligatures 23a and 23b might be affixed onto the central portion of the mesh tape 1 for handling easier, if necessary.

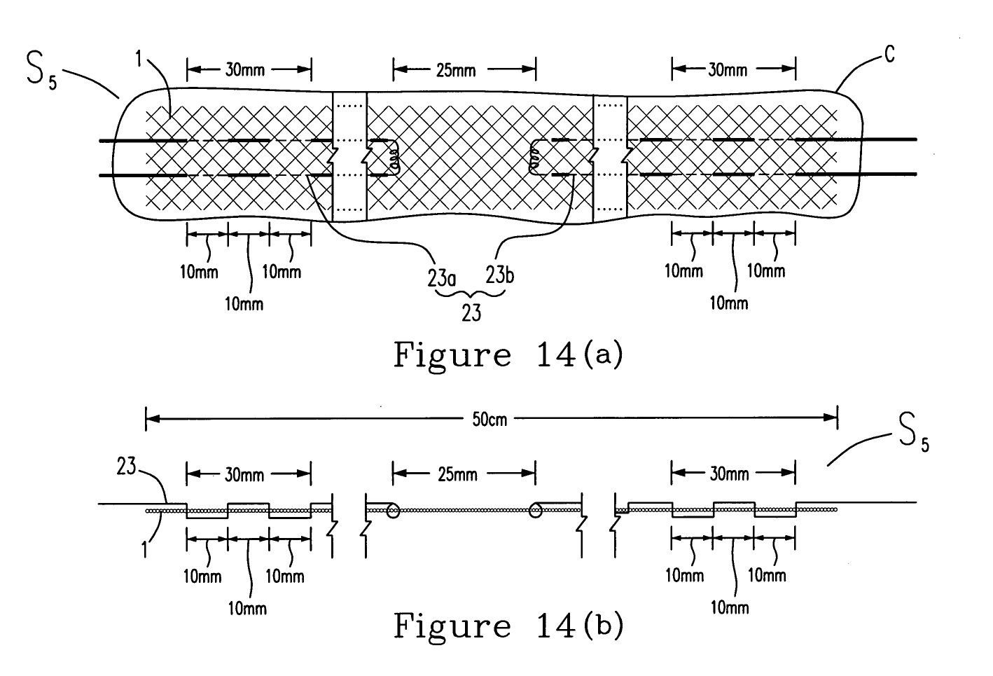

Please refer to FIGS. 14 (a) and (b), which are the schematic diagrams of the top view and the lateral view of the surgical mesh tape sling S₅ according to the third preferred embodiment of the present invention. As shown in FIGS. 14 (a) and (b), besides the mesh tape 1 and the ligatures 23a and 23b, the surgical mesh tape sling S₅ further includes a cover C for protecting and positioning. In general, the cover C would be removed from the mesh tape sling S₅ during the operation. The mesh tape 1 is made of a biocompatible material. The ligatures 23a and 23b are made of non-absorbable and non-adherent monofilament polypropylene. Since the ligatures 23a and 23b are respectively weaved into the mesh tape 1 from the ends and turned back helically nears the middle portion of the mesh tape 1, there are two loops formed from the ligatures 23a and 23b. However, in practice, sometimes, it is necessary to cut the middle portion of the mesh tape sling S₅ beneath the urethra for some special patients (such as urethral obstruction occurred after sling operation). Since the ligatures 23a and 23b could not be divided for they are not passing through the middle portion of the mesh tape 1, the adherent surgical mesh tape sling S₅ is still able to work. In addition, in order to position the mesh tape sling S₅ more easily, the central portion of the mesh tape sling S₅ might be colored or constructed with specific structures and dimensions. In addition, in order to position the mesh tape sling S₅ more easily, the central portion (such as the portion with the width of 25 mm shown in FIGS. 14 (a) and (b)) of the mesh tape sling S₅ might be colored or constructed with specific structures and dimensions. Further more, it also should be noted that it is also possible that the ligatures 23a and 23b might be affixed onto the central portion of the mesh tape 1 for handling easier, if necessary.

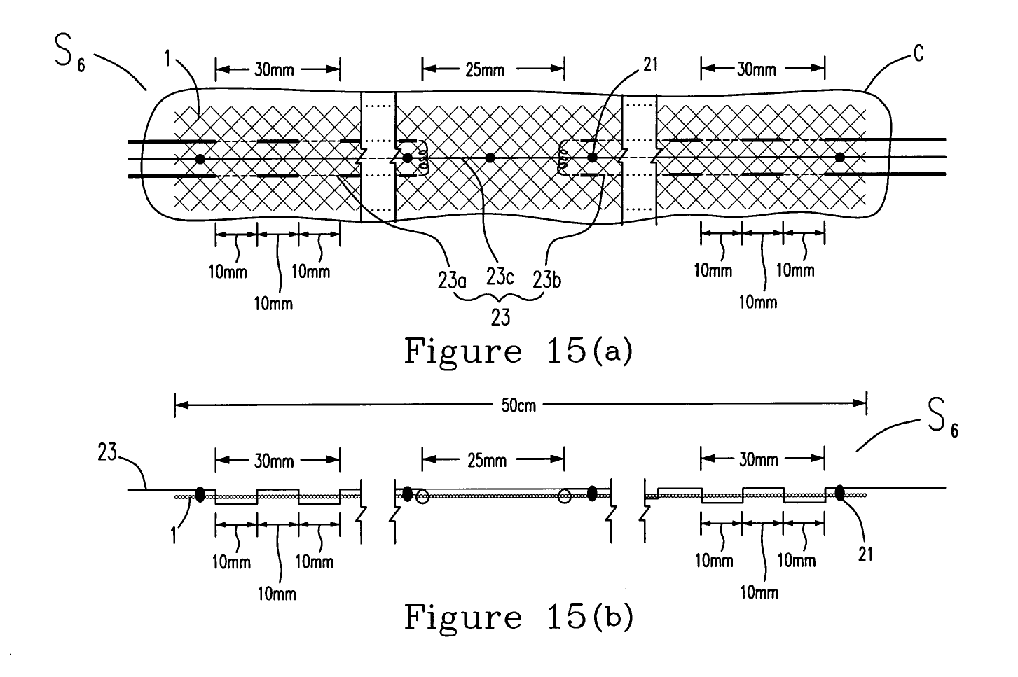

Please refer to FIGS. 15 (a) and (b), which are the schematic diagrams of the top view and the lateral view of the surgical mesh tape sling S₆ according to the fourth preferred embodiment of the present invention. As shown in FIGS. 15 (a) and (b), besides the mesh tape 1, the ligatures 23a and 23b, and the cover C, the surgical mesh tape sling S₆ further includes a third ligature 23c The mesh tape 1 is made of a biocompatible material. The ligatures 23a, 23b and 23c are made of non-absorbable and non-adherent monofilament polypropylene. Since the ligatures 23a and 23b are respectively weaved into the mesh tape 1 from the ends and turned back helically near the middle portion of the mesh tape 1, there are two loops formed from the ligatures 23a and 23b. The ligature 23c is embedded into the mesh tape 1 and with affixed knots 21 on the mesh tape 1 for keeping the mesh tape 1 from stretching deformity during a surgical procedure. In addition, in order to position the mesh tape sling S₆ more easily, the central portion (such as the portion with the width of 25 mm shown in FIGS. 15 (a) and (b)) of the mesh tape sling S₆ might be colored or constructed with specific structures and dimensions. Further more, it also should be noted that it is also possible that the ligatures 23a and 23b might be affixed onto the central portion of the mesh tape 1 for handling easier, if necessary.

In addition, the mesh tape slings S₃, S₄, S₅, and S₆ of the present invention have another unique character that the ligatures 23 of the mesh tape slings S₃, S₄, S₅, and S₆ can be used as a salvage procedure for a patient with recurrence. For instance, in a patient who suffers from a SUI associated with mild ISD, the mesh tape 1 alone may be provided as a satisfactory result. Therefore, we can make the knot 18 loose or leave the ligature 23 in place above the rectus fascia without tying during the anti-SUI procedure. The in placed ligature 23 could be tied through a small incision from the previous suprapubic incision 13 when the patient is recurrent from the previous surgery. The ligature 23 of the present invention could be used in a salvage procedure for a recurrent SUI through a minor procedure. Something important is that the salvage procedure can avoid the risk of the surgical complication on the bladder or urethra from a repeat anti-SUI procedure around tissue with scar and fibrosis formation.

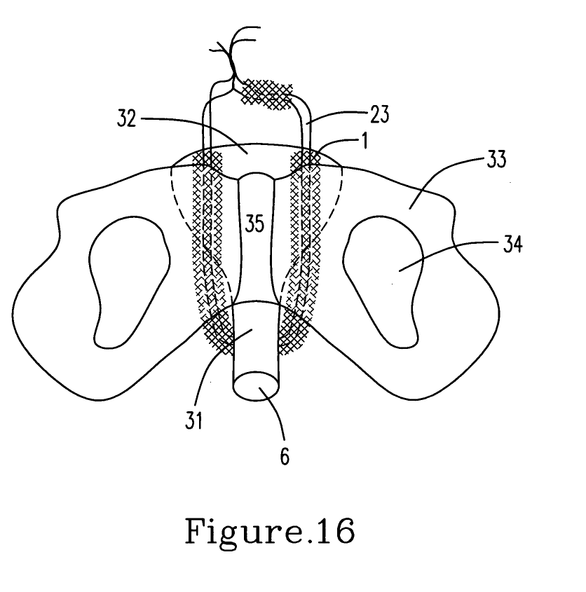

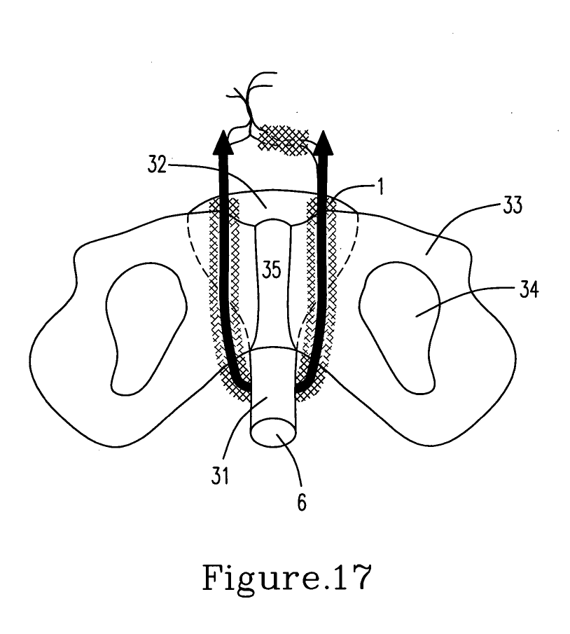

Please refer to FIGS. 16 and 17, which are the schematic diagrams showing the application of the surgical mesh tape sling S₃ of the present application in the suburethral sling anti-SUI procedure according to a preferred embodiment. In FIG. 16, the mesh tape 1, the ligature 23, the urethral meatus 6, the urethra 31, the bladder 32, the bladder 33, the obturator foramen 34, and the pubic symphysis 35 are illustrated. The non-absorbable monofilament ligature 23 will be pulled upward (shown in FIG. 17) on account of the contraction and elevation of the abdominal wall during coughing or abdominal straining and provide an active force to compress the urethra 31 accordingly. The mesh tape 1 is adhered and integrated into the tissue and a good hammock-like support under the midurethra is resulted accordingly. And, a passive dynamic kinking effect will be created at the midurethra by the mesh tape 1 during coughing or abdominal straining. However, it should be noted that the mesh tape sling S₃ in this embodiment is able to be replaced by the mesh tape slings S₄, S₅, or S₆.

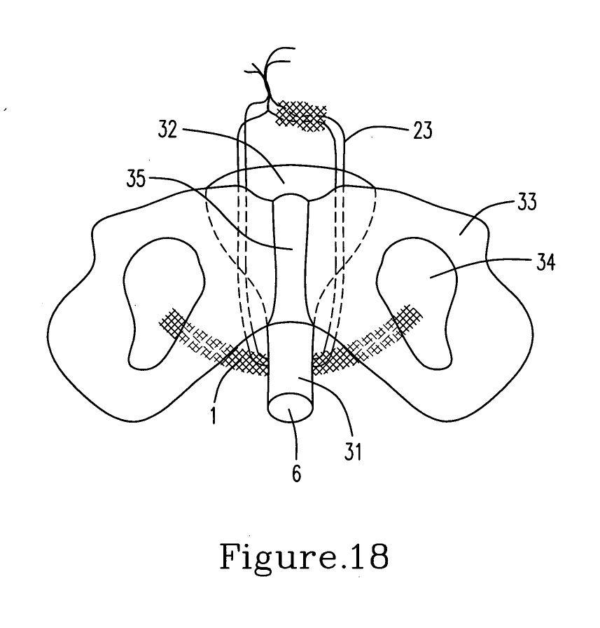

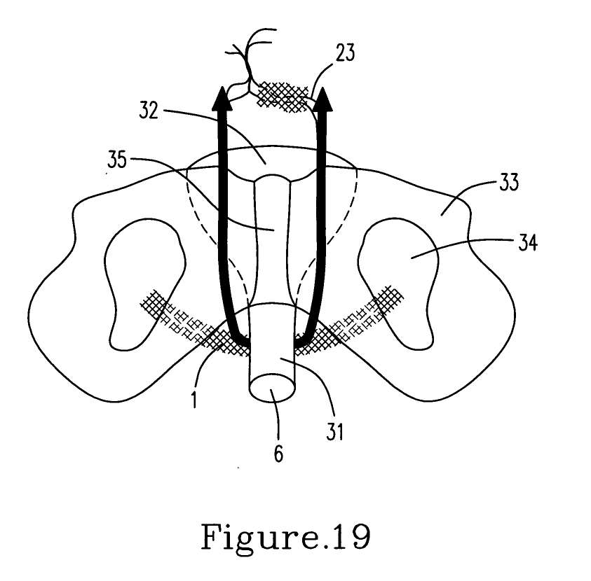

Please refer to FIGS. 18 and 19 which are the schematic diagrams showing the application of the surgical mesh tape sling S₅ in the transoburator suburethral sling anti-SUI procedure according to a preferred embodiment of the present invention. In FIG. 18, the mesh tape 1, the ligature 23, the urethral meatus 6, the urethra 31, the bladder 32, the bladder 33, the obturator foramen 34, and the pubic symphysis 35 are illustrated. The ligature 23 and the mesh tape 1 in the transobturator suburethral sling can also be used to provide two import elements for treating the SUI patients, i.e. an active force to compress the urethra 31 (shown in FIG. 19) and a passive kinking effect on the midurethra respectively, during coughing or abdominal straining. Similarly, the mesh tape sling S₅ in the present embodiment is able to be replaced by the mesh tape slings S₃, S₄ or S₆.

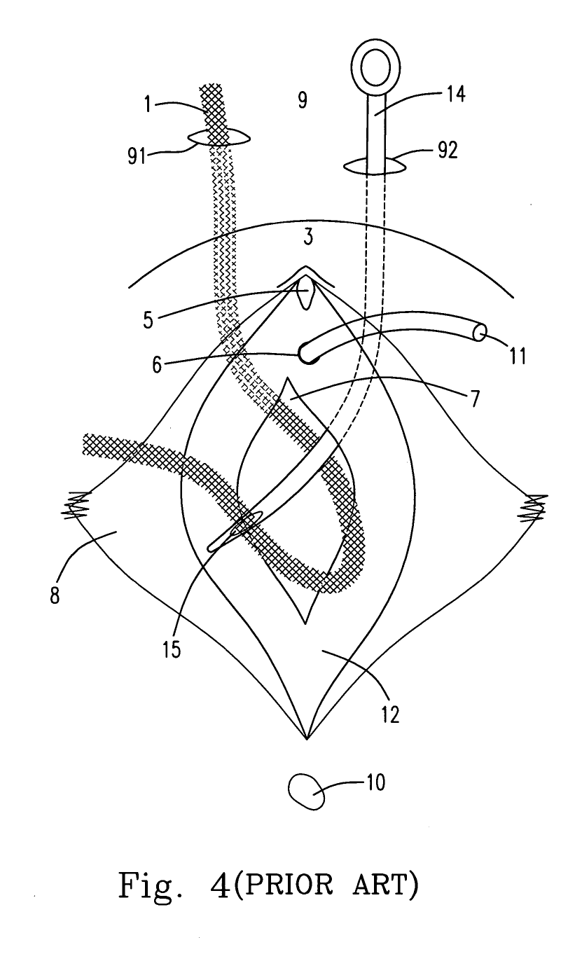

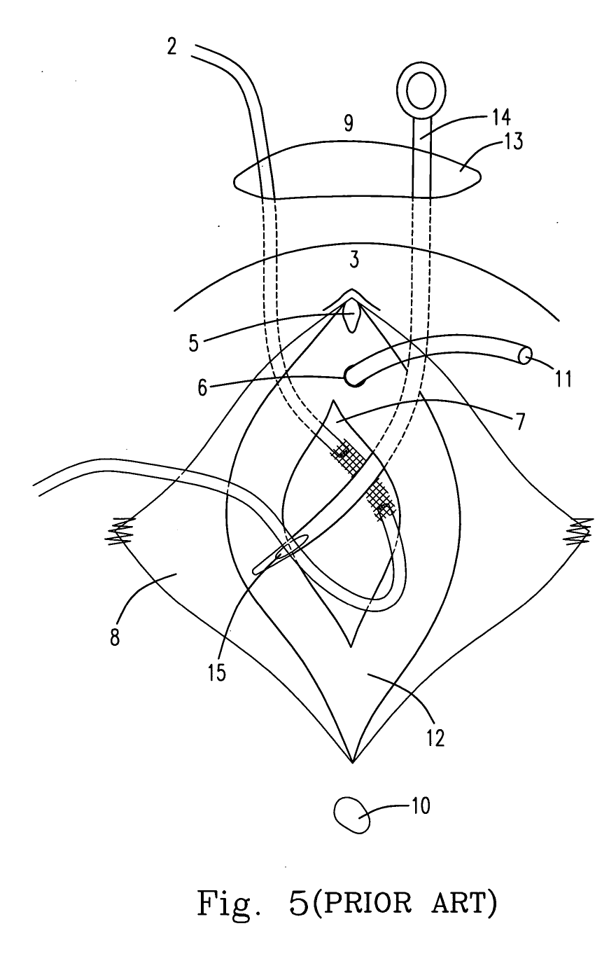

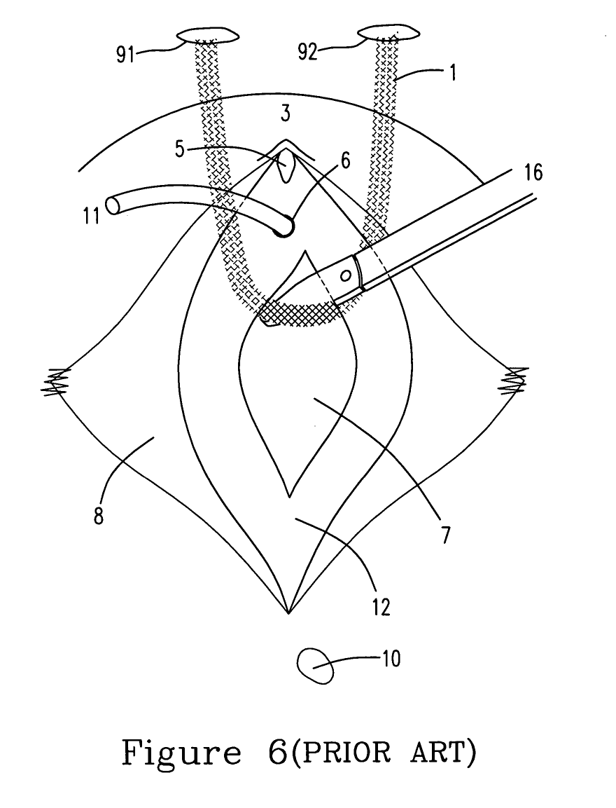

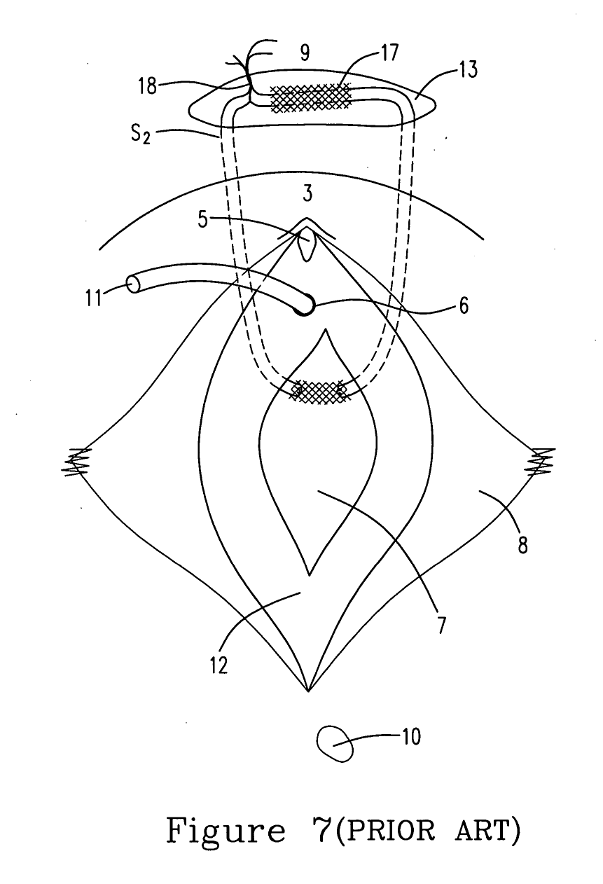

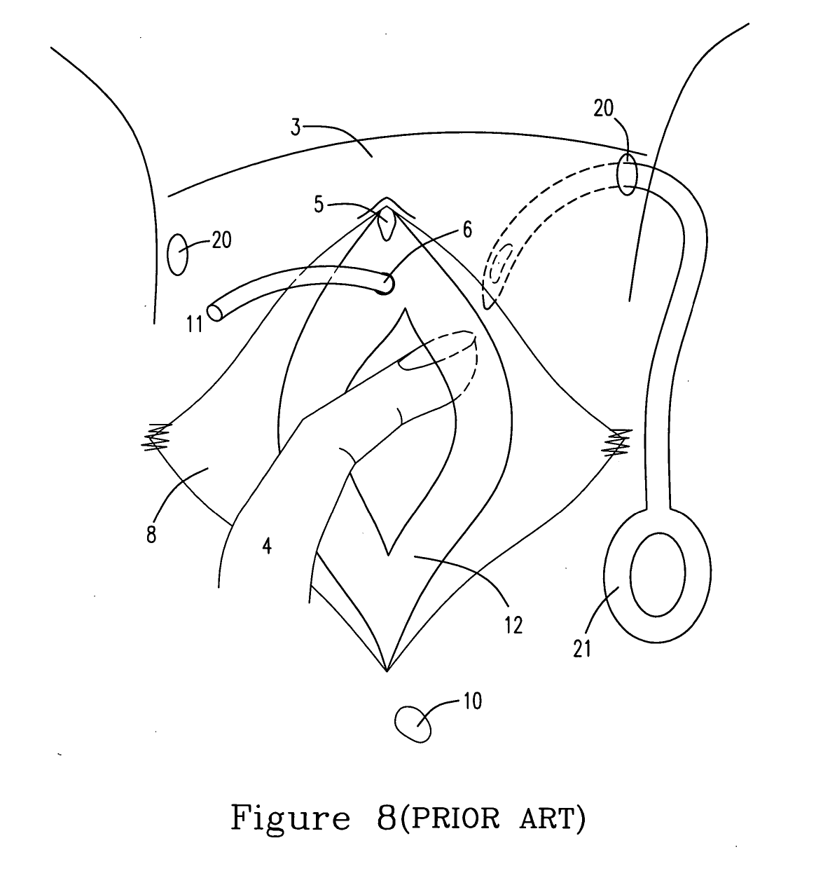

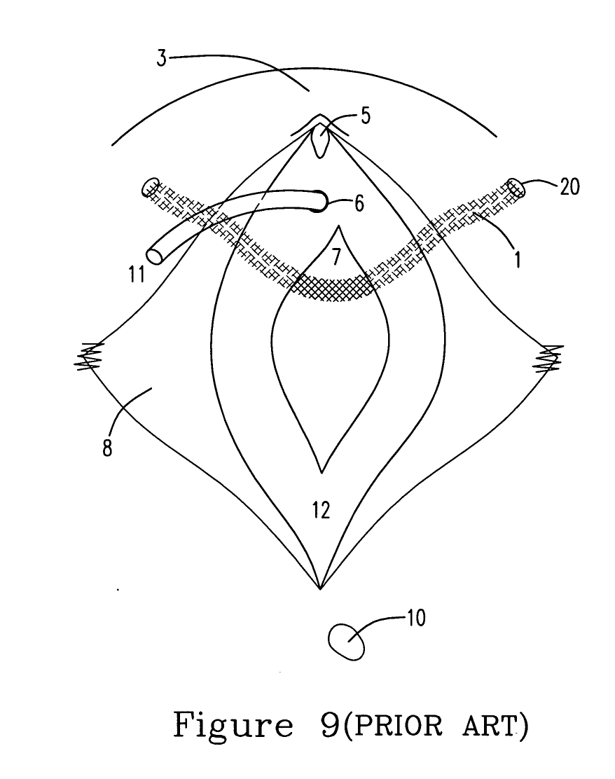

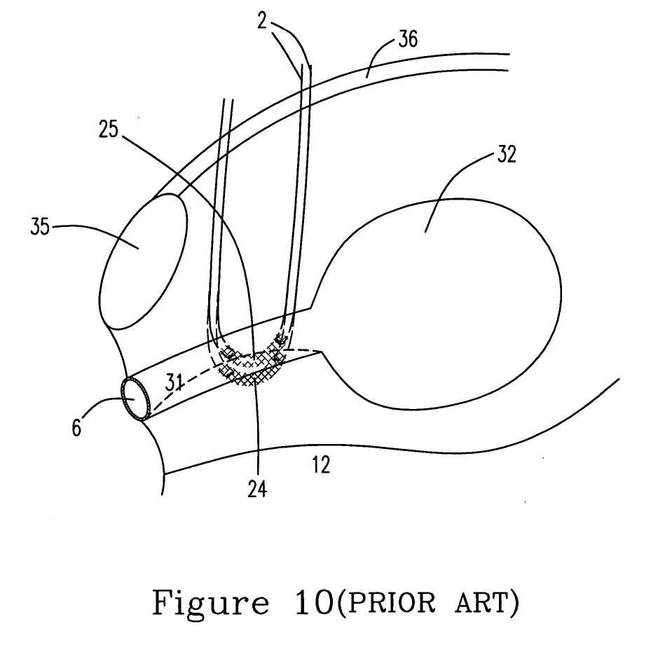

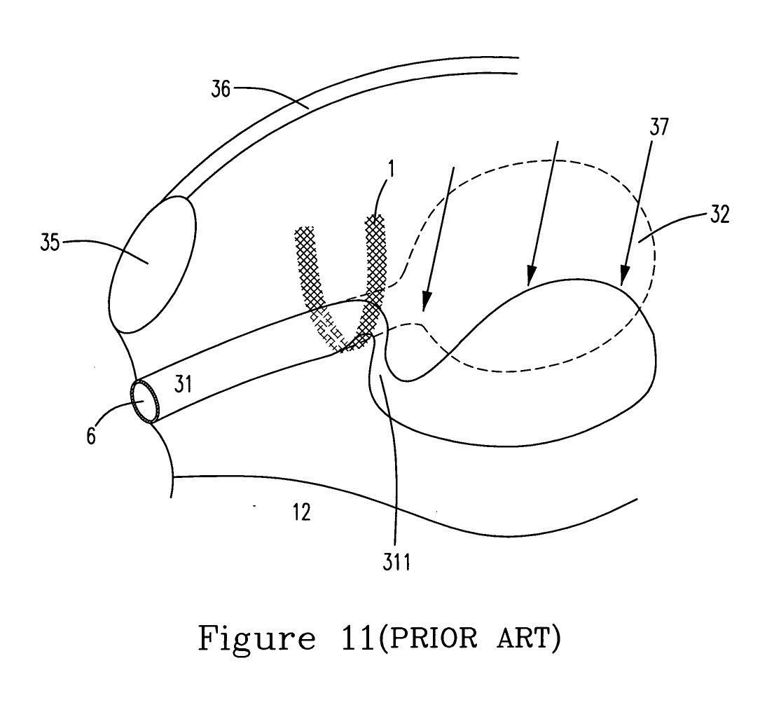

According to the present invention, since some ligatures (such as ligatures 23a and 23b in FIGS. 13, 14, 15 and 16) are weaved along the mesh tape without any affixed knot, the force of the abdominal wall movement during coughing or straining will pull the ligatures to against the urethra and urethral compression occurred. In addition, since the mesh tape is applied to create a new hammock-like structure to support the urethra and enhance the enforcement of the pubourethral ligaments for treating the SUI patient with ISD. The mesh tape 1 and the ligature 23 (including the ligatures 23a, 23b or 23c) of the mesh tape slings S₃, S₄, S₅ and S₆ can be placed together or separately. In the former, the mesh tape 1 and the ligatures are brought together by the carrier needle 14 from the vagina side up to the abdominal side via the retropubic space (similar to FIG. 4) In the latter, the mesh tape 1 is brought by the other carrier needle 21 from the vagina laterally to the genito-femoral folds via the obturator membrane (similar to FIG. 8) and the ligature 23 are brought separately by the carrier needle 14 from the vagina side up to the abdominal side via the retropubic space. (similar to FIG. 5) The mesh tape slings S₃, S₄, S₅, and S₆ of the present invention are able to provide both dynamic kinking and compression effects onto the urethra and to prevent urine leakage during coughing or abdominal straining for both the SUI patients with or without ISD. The mesh tape slings S₃ (FIG. 12), S₄ (FIG. 13), S₅ (FIG. 14), and S₆ (FIG. 15) in the present invention are respectively formed by combining the mesh tape 1 and the ligatures 23 together and are able to provide both mechanisms of dynamic kinking effect and automatic compression onto the urethra in one sling operation.

While the invention has been described in terms of what is presently considered to be the most practical and preferred embodiments, it is to be understood that the invention needs not be limited to the disclosed embodiment. On the contrary, it is intended to cover various modifications and similar arrangements included within the spirit and scope of the appended claims which are to be accorded with the broadest interpretation so as to encompass all such modifications and similar structures.

What is claimed is:

1. A surgical mesh tape, comprising:
a mesh; and
a holding ligature weaved into said mesh and capable of freely moving through said mesh.

2. The surgical mesh tape as claimed in claim 1, wherein said mesh is made of a biocompatible material.

3. The surgical mesh tape as claimed in claim 1, wherein said holding ligature is made of a monofilament of non-absorbable and non-adherent material.

4. The surgical mesh tape as claimed in claim 1, wherein said holding ligature is weaved into said mesh and without an affixed knot on said mesh.

5. The surgical mesh tape as claimed in claim 1, wherein said holding ligature is weaved into said mesh from one end of said mesh to the other opposite end of said mesh

6. The surgical mesh tape as claimed in claim 1, wherein said holding ligature is weaved into said mesh from a end of said mesh to a middle portion of said mesh and returned back to said end of said mesh.

7. The surgical mesh tape as claimed in claim 1, wherein a central portion of said mesh is marked by a color or a special structure.

8. The surgical mesh tape as claimed in claim 1, wherein said surgical tape is able of being delivered into a human body for a treatment purpose through different methods.

9. The surgical mesh tape as claimed in claim 1, wherein said surgical mesh tape is capable of being used for a patient having a stress urinary incontinence (SUI) and combined with an intrinsic sphincter deficiency (ISD).

10. The surgical mesh tape as claimed in claim 1, wherein said surgical mesh tape is capable of being used for a patient having stress urinary incontinence (SUI) but no serve intrinsic sphincter deficiency (ISD).

11. The surgical mesh tape as claimed in claim 1 further comprising a cover covering said mesh.

12. The surgical mesh tape as claimed in claim 11, wherein said cover is a removable cover.

13. The surgical mesh tape as claimed in claim 11, wherein said cover is used for positioning said mesh sling.

14. A surgical mesh tape, comprising;
a mesh sling;
at least a ligature weaved into said mesh sling and capable of freely moving through said mesh sling; and
an embedded ligature weaved into said mesh sling with affixed knots on said mesh.

15. The surgical mesh tape as claimed in claim 14, wherein said embedded ligature is used for keeping said mesh sling from stretching deformity during a surgical procedure.

16. The surgical mesh tape as claimed in claim 14 further comprising a cover covering said mesh sling.

17. A surgical mesh tape, comprising;
a mesh sling;
a cover covering said mesh sling; and
a ligature weaved into said mesh sling with an affixed knot on a central portion of said mesh sling.

18. The surgical mesh tape as claimed in claim 17, wherein said central portion has a width ranged from 1 mm to 50 mm.
